(19) United States
(12) Patent Application Publication
(10) Pub. No.: US 2018/0168568 A1
(43) Pub. Date: Jun. 21, 2018

(54) Needle Holder and Scissors Combination with Smooth Clamp Instrument

(71) Applicants: Angela Signorelli Ali, Jacksonville Beach, FL (US); Karim Nizar Ali, Jacksonville Beach, FL (US)

(72) Inventors: Angela Signorelli Ali, Jacksonville Beach, FL (US); Karim Nizar Ali, Jacksonville Beach, FL (US)

(21) Appl. No.: 15/836,865

(22) Filed: Dec. 9, 2017

Related U.S. Application Data
(60) Provisional application No. 62/435,707, filed on Dec. 16, 2016.

(51) Int. Cl. A61B 17/04; A61B 17/062

(52) U.S. Cl. CPC A61B 17/0467; A61B 17/285; A61B 17/062

(57) ABSTRACT

A combination needle holder, scissors, and smooth clamp instrument is disclosed. The instrument includes a first handle pivotally joined to a second handle defining opposite jaws at the distal end to form a gripping section and surface and at the rear of each jaw surfaces is a semicircular groove forming a circular groove when the handles are pivotally closed. The medical instrument has blades protruding out from each handle, situated at rear of the semi circular groove, and protruding from each handle in opposite direction. Adjacent to each protruding blade, situated in each handle, is a recessed groove which extends into the handle of the medical instrument and comprises the area to house and protect the opposite blade. Each handle of the present invention has a smooth surface rear of the blade and adjacent recessed groove, located on each handle, to form a smooth clamp when the handles are pivotally closed. The first and second handle comprises of finger loops at the proximal end of each handle of the instrument. The instrument also incorporates a locking mechanism situated in front of the finger loop of each handle to hold the needle in place, while gripping it in the jaws located distally of the instrument, when the handles are pivotally closed.

### CROSS-REFERENCE TO RELATED APPLICATIONS

This application claims the benefit of provisional patent application No. 62/435,707 filed 2016 Dec. 16 by present inventors.

### FIELD OF INVENTION

The present invention relates to a novel medical instrument combining the needle holder and scissors combination with smooth clamp to facilitate the wound repair by using sutures with suture needle and cutting the sutures with incorporated scissors without changing the instruments. This invention of needle holder and scissors combination incorporates a smooth clamp that can be used to clamp severed blood vessels or approximating the skin.

### BACKGROUND OF INVENTION

It has been a collective practice in medicine for clinicians or surgeons to use a separate instrument such as scissors to cut the sutures after the skin laceration, or tissue, is approximated with suture knot by needle holder and attached suture using the instrument needle holder. A clinician or surgeon must be ambidextrous and well-coordinated to draw the suture through the tissue with the needle holder and tying a knot and then cut the suture with a separate cutting instrument such as scissor. It is cumbersome for a physician, clinician, or surgeon to put down the needle holder and pick up a scissors each time to cut the suture. Then pick up the needle holder again to begin the next stitch. At times in emergency department, people can present with laceration that may require many stitches which can easily consume valuable time of a physician or clinician for a longer period of time while the other patients wait. Complex laceration may require an assistant to operate the scissors when placing multiple interrupted sutures.

In a normal practice of suturing a simple laceration, the curved needle, attached to the suture, is clamped by a needle holder with one hand by a clinician and passed through the both wound edges. The suture needle is then grasped by clinician's fingers or forceps using opposite hand. After which the clinician uses the needle holder to tie the suture knot and then places the needle holder on the instrument tray and pick up the scissors to cut the sutures above the knot.

Clinician then places the scissors on the instrument tray and picks up the needle holder to start the suturing process again. Therefore, for every suture knot placed to repair the wound, the clinician must repeatedly place needle holder on the instrument tray and pick up the scissors to cut the sutures. The procedure is time consuming and requires clinician to be present repairing the wound and not being able to attend to other sick patients while in the emergency department or urgent care setting when an assistant is not available. This timely procedure creates a distraction for clinician who is under the pressure of sick patients that are checking in the facility.

A simple or small laceration requires multiple sutures. However, frequently presenting lacerations are deep and may require absorbable sutures prior to closing the wound with removable sutures. This complicates the procedure further with multiple interchanging of the instruments and at times injuring the clinician or accidental dropping of the instruments on the floor or non sterile field and thus, opening up another sterile package and further compromising time.

In the emergency departments, or urgent care centers, where patients come in with fish hook injuries, the staff nurses or clinicians have to scramble and find pliers and different instruments to retract the fishing hook. Also at times while the clinician is repairing the wound laceration there might be a loose skin or skin tear that may need debridement or smooth the edges out. Clinicians have to place the needle holder and pick up the scissors to cut the skin and the scissors have to be used delicately to prevent piercing the tissue. However, to smooth the skin, they have to use forceps which can cause accidental piercing of the skin or may not be able to fold the skin correctly. Another instance in emergency department where drains, or tubes have to be clamped the only thing available is hemostat with jaws and clamping mechanism is not strong.

Currently stainless instruments used in emergency departments, surgical centers, and veterinarian clinic perform unique utility features and most of them are disposable. Thus, several instruments are used for single laceration repair and are being disposed after the wound repair. Thus constituting, hundreds of thousands of stainless steel instruments being disposed each day. This is substantial waste of metal and depletion of world resources. Invention of an instrument, comprising of multiple function, and utility features is needed so clinicians or allied health staff does not have to use multiple instruments saving time and resources and reducing metal waste. Furthermore, geographical regions or countries with limited resources, or military, can benefit from an invention of an instrument incorporating multiple function and thus benefiting civilization.

### SUMMARY OF THE INVENTION

One of the embodiment of the present invention of the medical instrument as disclosed combines the features of a needle holder and scissors combination and smooth clamp in one instrument. Medical instrument of the present invention has the first handle, which is pivotally joined, to the second handle at the pivot point by pivot attachment as is common to one skilled in the art such as by a screw, rivet, pin, or any other attachment means known to one skilled within the art. Each handle of the medical instrument has serrated, smooth, toothed, knurled, or textured jaws at the distal end so when the handles are pivotally closed they form a gripping section to grasp a suture needle or other item gripping it firmly. The first and second handle comprises of finger loops at the proximal end of each handle of the instrument. The instrument also incorporates a locking ratchet mechanism situated in front of the finger loop of each handle to hold the needle in place, while gripping it in the jaws located distally of the instrument, when the handles are pivotally closed without pressure on the handles by the operator.

Each handle of the medical instrument has an semi circular groove, located at the rear of the gripping surface of the jaw so when the handles are pivotally closed they form a circular groove. This embodiment of the surgical instrument facilitates to accommodate thicker diameter suture needles or to hold on to thicker fishing hook. One of the embodiments of the present invention is to easily aid in removing fishing hooks from the tissues.

The medical instrument as disclosed in the present invention, has blades protruding out from each handle, situated at rear of the semi circular groove, and protruding from each handle in opposite direction. Adjacent to each protruding blade, situated in each handle, is a recessed groove which extends into the handle of the medical instrument and comprises the area to encompass the opposite blade. So if the blade is protruding from same side of one handle and there is a recessed groove on the same side of the opposite handle to house and protect the blade. Therefore, when both handles of the medical instrument is pivotally closed, the blade from one handle will house and protected in recessed groove in the opposite handle.

Each handle of the present invention has a smooth surface rear of the blade and adjacent recessed groove, located on each handle, to form a smooth clamp when the handles are pivotally closed. This embodiment of the present invention facilitates to approximate the margins of wound or tissue or to clamp any blood vessels while minimizing tissue damage.

This embodiment of the present invention facilitates tying a suture knot and cutting the sutures without replacing the medical instrument. As the distal ends of the handles are pivoted towards each other, the jaw surfaces will be moved to a clamping position to grip a suture needle and the blade protruding out of each handle will be blocked by recessed groove on the opposite handle. The present invention of needle holder and scissors combination and smooth clamp instrument also minimizes accidental cutting of tissues while suturing as when the instrument is grasping the suture needle, while closed and handles pivoted towards each other, the scissors blades are housed and protected in recessed groove and flushed with instrument. Posterior to scissors blades will form a smooth clamping surface in front of the pivot. The rear of the pivot is the shank of each handle forming a finger loops and locking using ratchet mechanism, or any locking mechanism know to one skilled in art, in front of the finger loops, at the proximal end of the instrument.

The portion of each handle located between the jaw and the proximal end of jaw is formed with a circular groove and a scissors blades and as the distal ends are pivoted toward each other, the blades move into an overlapping cutting relation, so they can be used to cut the suture thread.

In one of the embodiment of the present invention a small transition zone separates the scissor blades from the jaw/gripping surfaces and the transition zone provides a visual separation between the scissors and the gripping jaws, which minimizes the possibility of the scissors accidentally grasping and severing the thread while tying a suture knot with the instrument. The semi circular groove, located rear of the gripping surface on each handle, also gives the operator of the instrument an indication of where the blade begins and the protrusion of the blade, from each handle, provides a visual area to bring the instrument over the sutures which facilitates the suture cut.

The exposed cutting edges become problematic. Given the delicacy of many anatomical structures, any sharp edges are extremely dangerous in a surgical instrument. And once damaged, some tissues, such as blood vessels, cannot be repaired easily, and may cause fatal consequences. One of the important embodiments of the present invention combines the needle holder and scissors and does not exposes any sharp edges until the jaws are spread far apart.

Another object of the present invention is that it can also be used for outdoor sports such as fishing or hunting. The front, or distally located jaws, can be used to grasp an object, and the circular groove formed by closing the jaws of the instrument can be used to grasp a thicker fishing hook. The protruded scissors blades can be used to cut fishing line. The formation of the smooth clamping surface of the instrument rear of the blades can be used to peel the skin of the fish or animal. Further embodiments of the present inventions will appear as the description proceeds.

### BRIEF DESCRIPTIONS OF DRAWINGS

FIG. 1 is a left side view of the present invention of the medical instrument that is pivotally closed and showing the scissor blades housed and protected in the recessed groove 14 and the instrument locked with ratchet mechanism and showing the distal jaws with gripping surface 6 and circular groove 8 and smooth clamping surface 12.

FIG. 2 is left side view of the present invention when it is pivotally open and showing the scissor blades forming a visual area.

FIG. 3 is a right side view of the present invention of the medical instrument that is pivotally closed and showing the scissor blades housed in the recessed groove 14 as shown in FIG. 2 and FIG. 4 and the instrument locked with ratchet mechanism and showing the distal jaws with gripping surface 6 as shown in FIG. 1 and FIG. 3 and circular groove 8 as shown in FIG. 1 and FIG. 3

FIG. 4 is right side view of the present invention of the medical instrument when it is pivotally open.

FIG. 5 is a left perspective view of the present invention of the medical instrument showing the blades showing the blades 10 and 10a and the recess groove 14 in FIG. 2 and FIG. 4 in which the blades 10 and 10a house when the medical instrument is pivotally closed. FIG. 5 is a left perspective view which also shows another preferred embodiment of the present invention and that is when the medical instrument is pivotally closed the surface 12a and 12b merge together to form a smooth clamp 12, as shown in FIG. 1 and FIG. 3.

FIG. 6 is a left side view of the present invention of the medial instrument when it is pivotally open and showing the scissor blades 10 and 10a, forming a visual area, and recessed groove 14.

FIG. 7 is right side view of the present invention of the medical instrument when it is pivotally open showing the handle 34 and 28, recessed groove 14, handle section 18 and 16, and blades 10a and 10.

FIG. 8 is a section taken along line 8-8 of FIG. 1 showing the blades 10 and 10 a as shown in FIG. 5 and housed and protected in recessed groove situated in opposite handles.

### DETAILED DESCRIPTION OF THE INVENTION

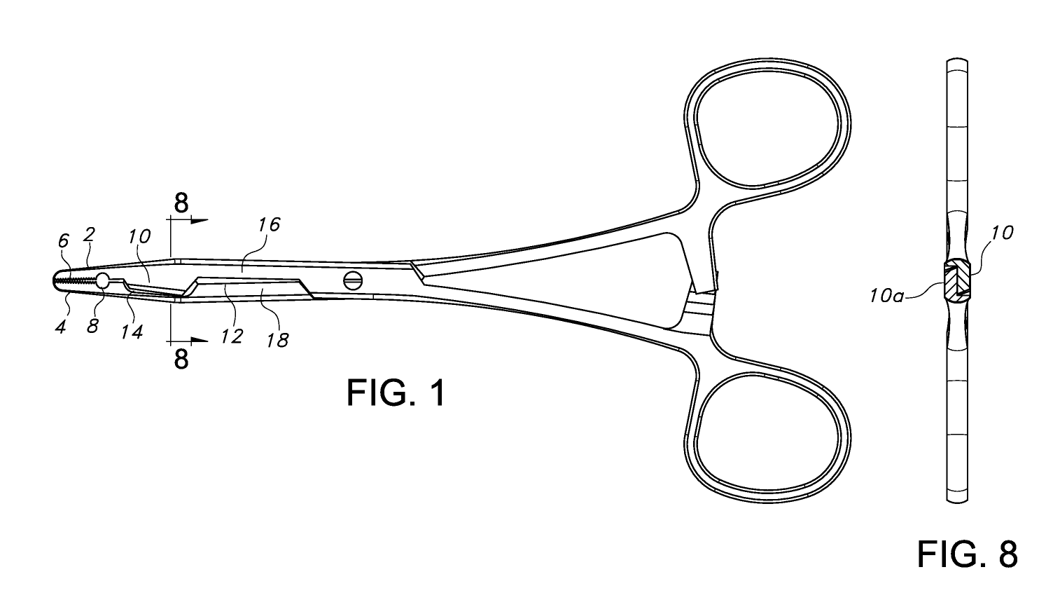

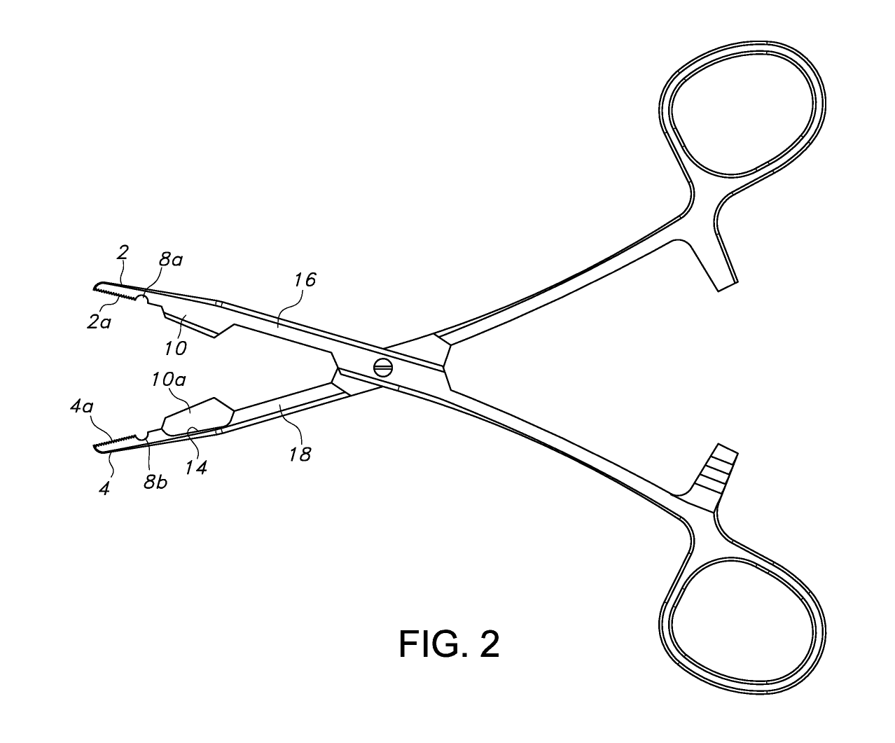

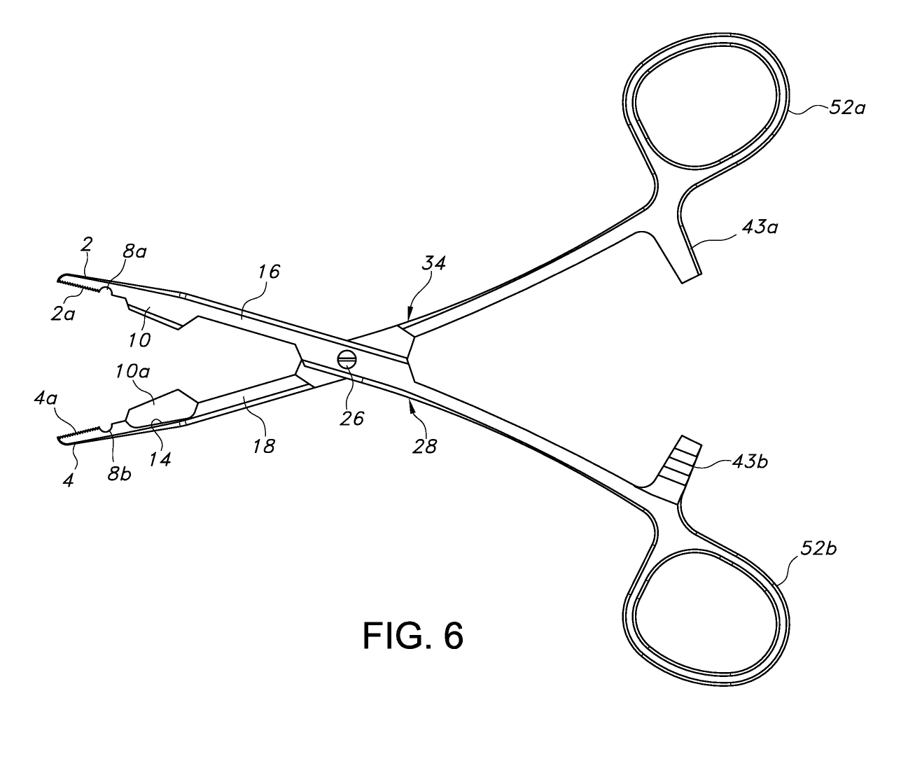

The present invention relates to a needle holder and scissor and smooth clamp combination instrument. The instrument comprises a first handle 34 and pivotally joined at 26, such as by a screw, rivet, pin, or any other attachment means known to one skilled within the art, to a second handle 28, as shown in FIG. 6. The handles 34 and 28 are pivotally connected together at a pivot point 26, as shown in FIG. 6, so that the distal ends of the handles can be pivoted between an open position, as shown in FIG. 2, and a closed position, as shown in FIG. 1.

With reference to FIG. 6, the first handle 34 and second handle 28 define a pair of opposing jaws 4 and 2 including a gripping surface 4a and 2a, respectively, to securely hold, for instance, a suture needle or other objects, when the handles are pivotally closed. The gripping section 6 of the instrument is formed when the handles are pivotally closed, as shown in FIG. 1. The gripping surface can be smooth, serrated, knurled, or textured to facilitate gripping object or suture needle.

The handles 34 and 28 of the present invention comprises of semicircular groove 8b and 8a, just aft of the gripping surface 4a and 2a, respectively, as shown in FIG. 6. When the handles are pivotally closed, the semicircular grooves 8b and 8a, in FIG. 6, form a circular groove 8, as shown in FIG. 1. The circular groove facilitates gripping larger or thicker object, or suture needle, or fishing hook. The semi circular groove, located rear of the gripping surface on each handle, also gives the operator of the instrument an indication of where the blade begins and the protrusion of the blade, from each handle, provides a visual area to bring the instrument over the sutures which facilitates to cut the sutures.

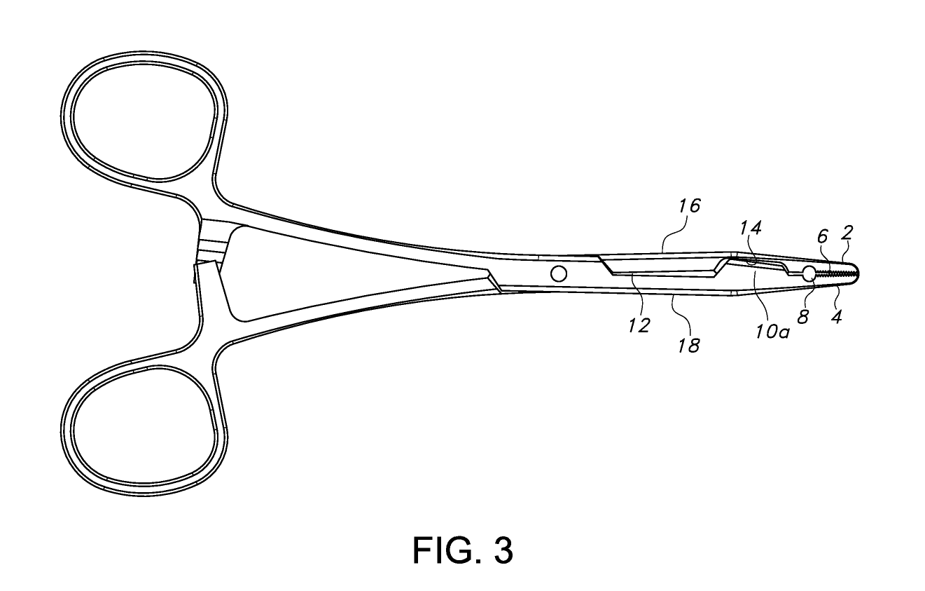

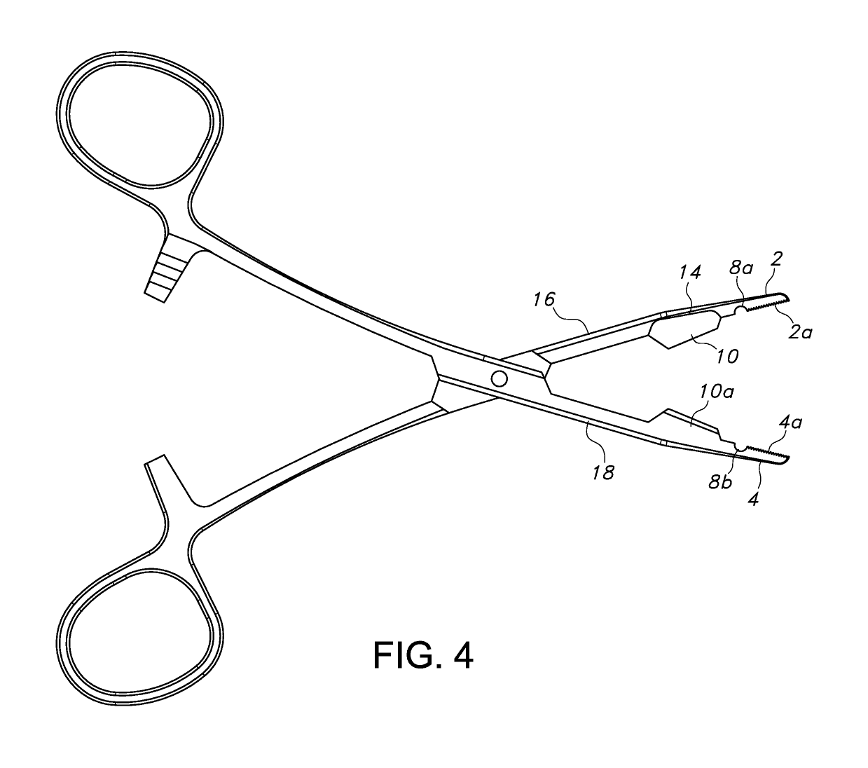

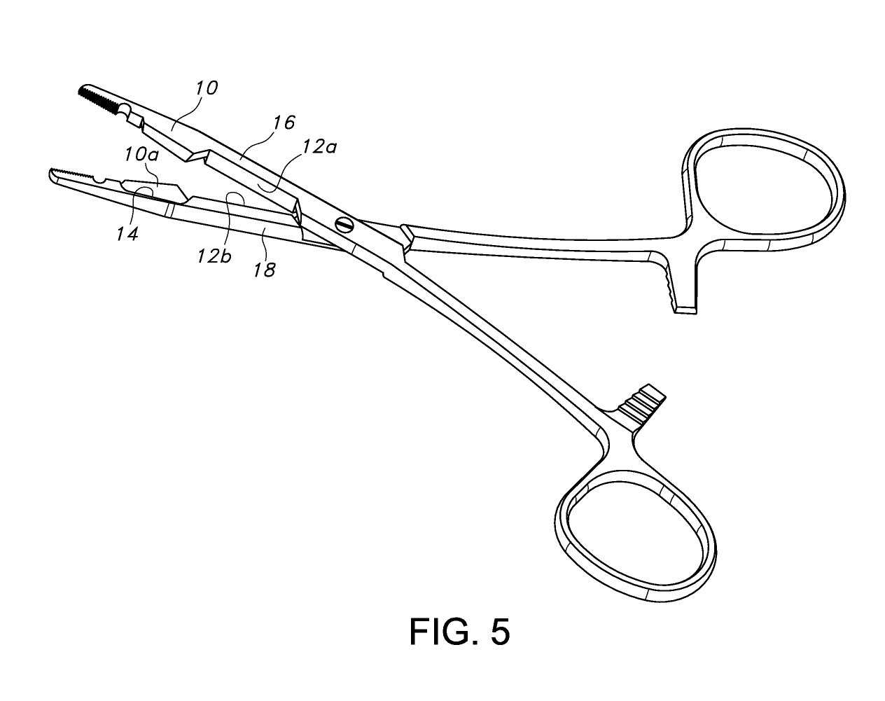

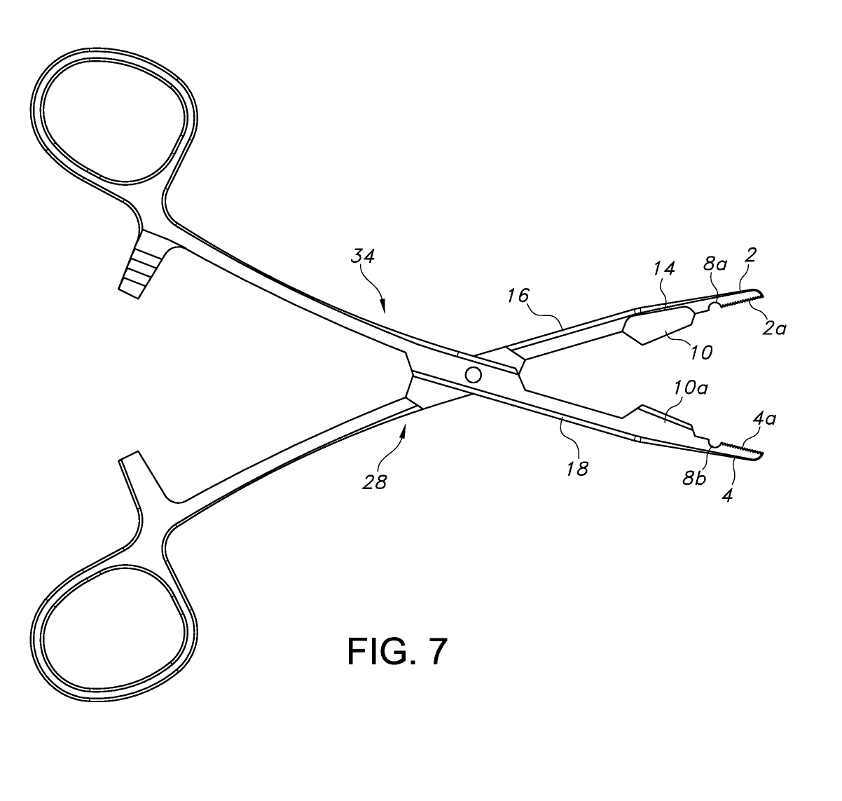

The medical instrument as disclosed in the present invention, has blades 10a and 10, as shown in FIG. 6, FIG. 4 and FIG. 5, protruding out from each handle 34 and 28, as shown in FIG. 6, and FIG. 7, situated at rear of the semi circular grooves 8b and 8a, respectively, and protruding from each handle in opposite direction. Adjacent to each protruding blade, situated in each handle, is a recessed groove 14, as shown in FIG. 6 and FIG. 4, which extends into the handle of the medical instrument and comprises the area to encompass the opposite blade. So if the blade is protruding from same side of one handle then there will be a recessed groove on the same side of the other handle to house the blade, as shown in FIG. 4 and FIG. 6, and also shown in FIG. 5. Therefore, when both handles of the medical instrument is pivotally closed, the blade from one handle will house and be protected in recessed groove 14 in the opposite handle, as shown in FIG. 1 and FIG. 3 and also in FIG. 4, FIG. 6, and FIG. 7.

In one of the embodiment of the present invention a small transition zone separates the scissor blades from the jaw surfaces and the transition zone provides a visual separation between the scissors and the gripping jaws, which minimizes the possibility of the scissors accidentally grasping and severing the thread while tying a suture knot with the instrument. The semi circular groove, located rear of the gripping surface on each handle, also gives the operator of the instrument an indication of where the blade begins and the protrusion of the blade, from each handle, provides a visual area to bring the instrument over the sutures which facilitates the suture cut. Another preferred embodiment of the present invention combines the needle holder and scissors and does not expose any sharp edges until the jaws are spread far apart as the blades are housed and protected in the recessed grooves.

Posterior to scissor blades is the section 18 and 16 of the handle 34 and 28, respectively, as shown in FIG. 6, in front of the pivot. The section of the handles 18 and 16 have smooth clamping surface 12b and 12a, respectively, as shown in FIG. 5. Therefore, when the handles are pivotally closed the clamping surfaces form a smooth clamp 12, as shown in FIG. 1 and FIG. 3. This embodiment of the present invention facilitates to approximate the margins of wound or tissue or to clamp any blood vessels while minimizing tissue damage or if used as an outdoor tool and can smooth the skin or clamp anything.

The rear of the pivot is the shank of each handle 34 and 28, forming a finger loops 52a and 52b and ratchet mechanism 43a and 43b in front of the finger loops, at the proximal end of each handle of the instrument, as shown in FIG. 6. Such finger loops make it easier for the user to operate the instrument and may be manufactured in various sizes to accommodate different finger diameters. When the handles 34 and 28, as shown in FIG. 6, are pivotally closed they are interlocked by the ratchet mechanism 43a and 43b, as shown in FIG. 6, using opposite teeth. This allows the user to hold the metal object, such as suture needle, securely in the gripping section of the jaws when the handles are pivotally closed and without any pressure from the hands of the operator. The locking mechanism can be of any design, which allows the jaws to remain in closed position while the user is gripping the object with the jaws when the handles are pivotally closed. The operator than manually pushes the finger loops outwardly through his thumb and finger to release the locking mechanism and release the object or suture needle grasped in the jaw and can pivotally open the handles while extending the thumb and finger upwards and downwards and hold the object or suture needle with fingers from the other hand or forceps.

The present invention permits a user to repair the wound laceration with suture needle and then cutting the suture material or thread with the same hand without the necessity of using a separate scissors instrument, or having an assistant cut the suture material with suture scissors. Thus the present invention allows for less wasted time. This is paramount in an emergency practice, or in veterinary surgery, or in the trauma practice. The present invention can also be used in outdoor activities such as fishing, and hunting. The present invention can also be used as kitchen or mechanical tool.

The instrument described in the present invention can be manufactured of any material having adequate strength and hardness to perform the intended purpose. The material can be the same as with conventional needle holder and scissors such as stainless steel, stainless steel alloys, titanium alloys, or composite materials, or plastics. The instrument of the present invention may be disposable or could be used several times after sterilization after each use. The instrument of present invention also allows other objects to be grasped by the jaw besides suture needles. This present invention is useful tool in order to save time in emergency, veterinary, or trauma surgery settings. This present invention is also useful kitchen tool to peel and cut the fruit and vegetable skins. The present invention is also useful in cutting fishing line and useful in other fishing and hunting activities.

What is claimed:

1. A medical instrument comprising:
a first handle which is pivotally joined, to the second handle at the pivot point by pivot attachment to define opposing jaws at a distal end of the medical instrument, each of said opposing jaws including a distal gripping surface, which has serrated, smooth, knurled, toothed, or textured surface;
a) each said handle of the medical instrument has a semi circular groove, located at the rear of the said gripping surface of the said jaw so when the said handles are pivotally closed they form a circular groove;
b) each blade, situated at the rear of the semicircular groove of each said handle, is protruding out from each said handles in opposite fashion and adjacent to each said blade, in each said handle, is a recessed groove, of same length as the opposite said blade, so the said blade protruding from each said handles will be housed in a said recessed groove of the opposite said handles and when the said handles are pivotally closed the said blades from each said handles are housed and protected by the said recessed grooves in each opposite said handles; and
c) each said handle of the medical instrument has a smooth surface rear of the said blade and adjacent to said recessed groove, located on each said handle, to form a smooth clamp when the said handles are pivotally closed.

2. The medical instrument of claim 1, wherein the medical instrument is a single use.

3. The medical instrument of claim 1, wherein the medical instrument is for multiple uses, after sterilization.

4. The medical instrument of claim 1, wherein the medical instrument is made of stainless steel.

5. The medical instrument of claim 1, wherein the medical instrument is made of organic polymer material.

6. The medical instrument of claim 1, wherein the medical instrument can be used in outdoor activities.

7. The medical instrument of claim 1, wherein the medical instrument can be used as a mechanical tool.

8. The medical instrument of claim 1, wherein the medical instrument can be used as a kitchen tool.

9. The medical instrument of claim 1, wherein the medical instrument is made of tungsten carbide.
